(19) United States
(12) Patent Application Publication
(10) Pub. No.: US 2026/0306606 A1
(43) Pub. Date: Oct. 8, 2026

(54) Pharmaceutical Composition for Vaccination against Cytomegalovirus Infections

(71) Applicant: INSTAR TECHNOLOGIES A.S., Liberec (CZ)

(72) Inventors: Denisa STRÁNSKÁ, Liberec (CZ); Pavel BOŠTIK, Liberec (CZ); Gyan SAGAR, Liberec (CZ); Sylva JANOVSKÁ, Liberec (CZ); Radek ŠLEHA, Liberec (CZ)

(73) Assignee: INSTAR TECHNOLOGIES A.S., Liberec (CZ)

(21) Appl. No.: 19/476,593

(22) PCT Filed: Apr. 20, 2024
(86) PCT No.: PCT/IB2024/053866

§ 371 Date: Oct. 17, 2025

(30) Foreign Application Priority Data
Apr. 21, 2023 (CZ) PV 2023-162

(51) Int. Cl. A61K 39/12; A61K 9/00; A61K 9/70; A61K 39/00; A61K 47/02; A61K 47/34; A61P 37/04

(52) U.S. Cl. CPC A61K 39/12; A61K 9/006; A61K 9/70; A61K 47/02; A61K 47/34; A61P 37/04; A61K 2039/542

(57) ABSTRACT

The present invention relates to the field of prevention of herpes infections, in particularly to a pharmaceutical composition for oromucosal administration for vaccination against cytomegalovirus infections and the use of cytomegalovirus glycoprotein B. The pharmaceutical composition comprises, as an active ingredient, cytomegalovirus glycoprotein B, which is releasably contained in the pharmaceutical composition in a carrier for oromucosal administration of physiologically active agents, comprising at least one flexible active layer of polymeric nanofibers in which cytomegalovirus glycoprotein B is releasably contained, and at least one cover layer of polymeric nanofibers.

### CROSS-REFERENCE TO RELATED APPLICATIONS

This application claims the benefit of priority to European Patent Application No. 23020210.3, filed on May 4, 2023, the contents of which are incorporated herein by reference in its entirety as if set forth herein.

### BACKGROUND OF THE INVENTION

Cytomegalovirus (CMV) is a ubiquitous virus belonging to the family Herpesviridae, which infects 45 to 100% of people worldwide in different parts of the world [1]. It occurs in a latent state in healthy people and maybe a common cause of disease in immunosuppressed patients [2]. In a study conducted on two hundred and eighty-three students of the Faculty of Science, UK, the prevalence of cytomegalovirus was 52.2% in all subjects, 49.7% in females, and 58.1% in males [1].

The structure of cytomegalovirus, or its virion, consists of linear double-stranded DNA, which is enclosed by a protein capsid. The lipid bilayer, present above the protein capsule, is enriched with a large number of viral glycoproteins. The glycoprotein B or gB protein encoded by the UL55 gene is the most important glycoprotein and is the main component of the envelope of the cytomegalovirus. The gB protein is also an important antigen against which specific antibodies are directed when the cytomegalovirus enters the host body.

Treatment of infections caused by cytomegaloviruses is based on the administration of virostatics, which are usually taken orally or intravenously. The most commonly used antiviral drug is ganciclovir or valacyclovir. As a long-term maintenance therapy, most patients prefer oral administration of ganciclovir in 1000 mg capsules three times a day to intravenous administration for the treatment of cytomegalovirus retinitis. To achieve efficacy similar to ganciclovir, a higher dose of valacyclovir is needed [3], [4], which could lead to several disadvantages as both virostatics have a relatively wide range of side effects that is proportional to the increase of dosage. However, in addition, another disadvantage of this administration is its low bioavailability [3].

Most pathogens encountered by the immune system enter the body through the mucosal surfaces of the respiratory, gastrointestinal, or urogenital tract. These are the most extensive surfaces of the human body that are in contact with the external environment. The mucosal immune system is composed of lymphoid tissue that is enriched with immune cells. Mucosal lymphoid tissues play an important role in the generation of lymphocytes, including specific IgA effector B cells, and memory B cells and T cells. This includes active lymphocyte proliferation, local production of certain cytokines, and continuous intercellular signaling. Capture of the antigen and initiation of the immune response first requires access to the antigen.

Therefore, there is a need for the development of oral or mucosal vaccines to prevent infectious disease at the point of contact/entry and include oral immunization platform that allows efficient delivery of antigens and adjuvants. Although oral vaccines have been available on the market for some time, the development of oromucosal vaccines is still needed. In particular, to ensure sufficient and well-defined contact of vaccine agents with mucosal surfaces, which is still a problem with existing types of products.

The available literature provides information on the development of CMV vaccine at different drug development stages, from preclinical to clinical trials. Different approaches include—recombinant protein subunit vaccines; poxviral and alphaviral vector subunit vaccines; DNA vaccines; live attenuated vaccines; dense body vaccines; passive vaccination strategies based on adoptive transfer of CMV-specific T cells and neutralizing immunoglobulins (IgG). Although potential target structures eliciting an adequate immune response have been identified in the past, the possibility of preventive vaccination has not yet been successfully addressed.

The aforementioned shortcomings establish the need to create a new pharmaceutical composition for oromucosal administration, in particular for vaccination against cytomegalovirus infections, which would exhibit higher bioavailability compared to other orally administered vaccines. The introduction/inclusion of oromucosal administration would stimulate mucosal immunity, thus preventing infection at the point of entry or its reactivation, and would reduce the current dependence on virostatics which is coupled with numerous side effects.

The present invention addresses these and other needs in the art.

### SUMMARY

These and other embodiments, features, and advantages will become apparent to those skilled in the art when taken with reference to the following more detailed description of various exemplary embodiments of the present disclosure in conjunction with the accompanying drawings.

According to frequently included embodiments, there is provided a pharmaceutical composition for oromucosal administration, comprising: at least one coating layer comprising a water-insoluble, pharmaceutically acceptable polymer, and a conductive agent; an active layer comprising a water-soluble and pharmaceutically acceptable polymer, a conductive agent, and cytomegalovirus glycoprotein B as the active ingredient, wherein the active layer is flexible and moldable.

According to often included embodiments, there is provided a pharmaceutical composition for oromucosal administration as above, wherein cytomegalovirus glycoprotein B is present at 2 to 250 μg, at 21 to 40 μg, at 41 to 60 μg, at 61 to 80 μg, at 81 to 100 μg, at 101 to 120 μg, at 121 to 160 μg, at 161 to 180 μg, at 181 to 200 μg, at 201 to 200 μg, at 221 to 240 μg, at 241 to 250 μg.

According to often included embodiments, there is provided a pharmaceutical composition for oromucosal administration as above, wherein cytomegalovirus glycoprotein B is present at 2 to 85 μg, at 86 to 170 μg, at 171 to 250 μg.

According to frequently included embodiments, there is provided a pharmaceutical composition for oromucosal administration as above, wherein the active layer comprises polymeric nanofiber formed by electrospinning.

According to often included embodiments, there is provided a pharmaceutical composition for oromucosal administration as above,

According to frequently included embodiments, there is provided a pharmaceutical composition for oromucosal administration as above, wherein cytomegalovirus glycoprotein B is releasably deposited in the polymer nanofiber.

According to often included embodiments, there is provided a pharmaceutical composition for oromucosal administration as above, wherein the at least one coating layer comprises polymeric nanofibers formed by electrospinning.

According to frequently included embodiments, there is provided a pharmaceutical composition for oromucosal administration as above, wherein the conductive agent is an acid, a base, a neutral salt, an acidic salt, or a basic salt.

According to often included embodiments, there is provided a pharmaceutical composition for oromucosal administration as above, wherein the acid is phosphoric acid, formic acid, acetic acid, citric acid, lactic acid, acetic acid, ascorbic acid, malic acid, gluconic acid, glutamic acid, hydrochloric acid, sulfuric acid, succinic acid, tartaric acid, or butyric acid.

According to frequently included embodiments, there is provided a pharmaceutical composition for oromucosal administration as above, wherein the base is sodium hydroxide or potassium hydroxide.

According to often included embodiments, there is provided a pharmaceutical composition for oromucosal administration as above, wherein the neutral salt is sodium chloride, potassium chloride, arginine hydrochloride, triethanolamine, or tetraethylammonium bromide.

According to frequently included embodiments, there is provided a pharmaceutical composition for oromucosal administration as above, wherein the acidic salt is sodium hydrogen sulfate, mono- or di-potassium sulfate, or mono- or di-sodium phosphate.

According to often included embodiments, there is provided a pharmaceutical composition for oromucosal administration as above, wherein the basic salt is sodium (bi) carbonate or sodium acetate.

According to frequently included embodiments, there is provided a pharmaceutical composition for oromucosal administration as above, wherein water-soluble and pharmaceutically acceptable polymer is polyethylene glycols (PEG), polyethylene oxides (PEO), copolymers of polyethylene oxide with other alkylene oxides, copolymers of PEG and polypropylene oxide or polybutylene oxide, polyvinyl alcohol, polyvinylpyrrolidones (PVP), albumin, dextran, hyaluronic acid, alginates, carrageenan, chitosan, gelatin, collagen, zein and derivatives, polyacrylic acid, cellulose derivatives such as sodium carboxymethyl cellulose, hydroxyethyl cellulose (HEC), hydroxypropyl cellulose (HPC), hydroxypropyl methylcellulose (HPMC), hydroxypropyl methylcellulose phthalate, cellulose acetate phthalate, or oxidized cellulose.

According to often included embodiments, there is provided a pharmaceutical composition for oromucosal administration as above, wherein water insoluble and pharmaceutically acceptable polymer is acetylcellulose, methylcellulose, ethylcellulose, noncrystalline cellulose, acrylates, methacrylates, alkylacrylates and copolymers thereof, (meth)acrylic acid, (meth)acrylamides, hydroxyethyl (meth)acrylates, poly(alpha-hydroxy acids), poly(c-caprolactone), poly(lactide-co-glycolide), poly(alpha-aminoacids) and its copolymers, or polyurethane.

According to frequently included embodiments, there is provided a pharmaceutical composition for oromucosal administration as above, wherein water-insoluble and pharmaceutically acceptable polymer is a biodegradable polymer.

According to often included embodiments, there is provided a pharmaceutical composition for oromucosal administration as above, wherein the biodegradable polymer is polycaprolactone, polylactic acid, poly(lactide-co-glycolide), or polyglycolic acid.

According to frequently included embodiments, there is provided a pharmaceutical composition for oromucosal administration as above, the pharmaceutical composition further comprises a wetting agent or a penetration enhancer.

According to often included embodiments, there is provided a pharmaceutical composition for oromucosal administration as above, wherein the wetting agent or penetration enhancer is anionic surfactants, cationic surfactants, ionic surfactants, fatty acids and derivatives, sulfoxides, alcohols, chelating agents, bile salts and derivatives, cyclodextrins, polyvinylpyrrolidone, lactose, triacetin, and/or menthol.

According to frequently included embodiments, there is provided a use of the pharmaceutical composition as above in the prevention of cytomegalovirus infections.

According to often included embodiments, there is provided a use of the pharmaceutical composition as above in the treatment of cytomegalovirus infections.

According to often included embodiments, there is provided a use of the pharmaceutical composition as above, wherein the pharmaceutical composition is administered to an oromucosal membrane of a subject.

According to frequently included embodiments, there is provided a use of the pharmaceutical composition as above, wherein the pharmaceutical composition is administered to a subject with a prime administration followed by two booster administrations.

According to often included embodiments, there is provided a use of the pharmaceutical composition as above, wherein the prime administration is administered on day 1, and the two booster administrations are administered on day 14 and day 28.

According to frequently included embodiments, there is provided a use of the pharmaceutical composition as above, wherein the prime administration is administered on day 1, followed by first booster on at least 7 days thereafter, followed by second booster on at least 7 days thereafter.

According to often included embodiments, there is provided a use of the pharmaceutical composition as above, wherein the pharmaceutical composition of claim 1 is administered to a subject with a prime administration followed by at least one booster administration, and each booster administration is on at least 7 days after the prior administration.

According to frequently included embodiments, there is provided a use of cytomegalovirus glycoprotein B for the production of a vaccine for oromucosal vaccination against cytomegalovirus infections.

According to often included embodiments, there is provided a method to manufacture a pharmaceutical composition for oromucosal administration, comprising: combining a water-insoluble and pharmaceutically acceptable polymer and a conductive agent into water to form a first solution; electrospinning the first solution; collecting nanofibers produced from electrospinning of the first solution on a mat, the collected nanofibers forming a coating layer; combining cytomegalovirus glycoprotein B, a water-soluble and pharmaceutically acceptable polymer, and a conductive agent into water to form a second solution; electrospinning the second solution; and collecting nanofibers produced from electrospinning of the second solution on top of the coating layer.

According to frequently included embodiments, there is provided a method to manufacture a pharmaceutical composition for oromucosal administration as above, wherein the water-insoluble and pharmaceutically acceptable polymer is acetylcellulose, methylcellulose, ethylcellulose, noncrystalline cellulose, acrylates, methacrylates, alkylacrylates and copolymers thereof, (meth)acrylic acid, (meth)acrylamides, hydroxyethyl (meth)acrylates, poly(alpha-hydroxy acids), poly(c-caprolactone), poly(lactide-co-glycolide), poly(alpha-aminoacids) and its copolymers, or polyurethanes.

According to often included embodiments, there is provided a method to manufacture a pharmaceutical composition for oromucosal administration as above, wherein the water-soluble and pharmaceutically acceptable polymer is polyethylene glycols (PEG), polyethylene oxides (PEO), copolymers of polyethylene oxide with other alkylene oxides, copolymers of PEG and polypropylene oxide or polybutylene oxide, polyvinyl alcohol, polyvinylpyrrolidones (PVP), albumin, dextran, hyaluronic acid, alginates, carrageenan, chitosan, gelatin, collagen, zein and derivatives, polyacrylic acid, cellulose derivatives such as sodium carboxymethyl cellulose, hydroxyethyl cellulose (HEC), hydroxypropyl cellulose (HPC), hydroxypropyl methylcellulose (HPMC), hydroxypropyl methylcellulose phthalate, cellulose acetate phthalate, or oxidized cellulose.

According to frequently included embodiments, there is provided a method to manufacture a pharmaceutical composition for oromucosal administration as above, wherein the conductive agent is an acid, a base, a neutral salt, an acidic salt, or a basic salt.

According to often included embodiments, there is provided a method to manufacture a pharmaceutical composition for oromucosal administration as above, wherein the acid is phosphoric acid, formic acid, acetic acid, citric acid, lactic acid, acetic acid, ascorbic acid, malic acid, gluconic acid, glutamic acid, hydrochloric acid, sulfuric acid, succinic acid, tartaric acid, or butyric acid.

According to frequently included embodiments, there is provided a method to manufacture a pharmaceutical composition for oromucosal administration as above, wherein the base is sodium hydroxide or potassium hydroxide.

According to often included embodiments, there is provided a method to manufacture a pharmaceutical composition for oromucosal administration as above, wherein the neutral salt is sodium chloride, potassium chloride, arginine hydrochloride, triethanolamine, or tetraethylammonium bromide.

According to frequently included embodiments, there is provided a method to manufacture a pharmaceutical composition for oromucosal administration as above, wherein the acidic salt is sodium hydrogen sulfate, mono- or di-potassium sulfate, mono- or di-sodium phosphate.

According to often included embodiments, there is provided a method to manufacture a pharmaceutical composition for oromucosal administration as above, wherein the basic salt is sodium (bi) carbonate or sodium acetate.

According to frequently included embodiments, there is provided a method to manufacture a pharmaceutical composition for oromucosal administration as above, further comprising combining a wetting agent and/or a penetration enhancer into the first and/or the second solution.

According to often included embodiments, there is provided a method to manufacture a pharmaceutical composition for oromucosal administration as above, wherein the wetting agent or penetration enhancer is anionic surfactants, cationic surfactants, ionic surfactants, fatty acids and derivatives, sulfoxides, alcohols, chelating agents, bile salts and derivatives, cyclodextrins, polyvinylpyrrolidone, lactose, triacetin, and/or menthol.

According to frequently included embodiments, a solution for electrospinning is provided, the solution comprises: a water soluble and pharmaceutically acceptable polymer, a conductive agent, and cytomegalovirus glycoprotein B.

According to often included embodiments, there is provided a solution for electrospinning as above, wherein the water insoluble and pharmaceutically acceptable polymer is acetylcellulose, methylcellulose, ethylcellulose, noncrystalline cellulose, acrylates, methacrylates, alkylacrylates and copolymers thereof, (meth)acrylic acid, (meth)acrylamides, hydroxyethyl (meth)acrylates, poly(alpha-hydroxy acids), poly(c-caprolactone), poly(lactide-co-glycolide), poly(alpha-aminoacids) and its copolymers, or polyurethane.

According to frequently included embodiments, there is provided a solution for electrospinning as above, wherein the water soluble and pharmaceutically acceptable polymer is polyethylene glycols (PEG), polyethylene oxides (PEO), copolymers of polyethylene oxide with other alkylene oxides, copolymers of PEG and polypropylene oxide or polybutylene oxide, polyvinyl alcohol, polyvinylpyrrolidones (PVP), albumin, dextran, hyaluronic acid, alginates, carrageenan, chitosan, gelatin, collagen, zein and derivatives, polyacrylic acid, cellulose derivatives such as sodium carboxymethyl cellulose, hydroxyethyl cellulose (HEC), hydroxypropyl cellulose (HPC), hydroxypropyl methylcellulose (HPMC), hydroxypropyl methylcellulose phthalate, cellulose acetate phthalate, or oxidized cellulose.

According to often included embodiments, there is provided a solution for electrospinning as above, wherein the conductive agent is an acid, a base, a neutral salt, an acidic salt, or a basic salt.

According to frequently included embodiments, there is provided a solution for electrospinning as above, wherein the acid is phosphoric acid, formic acid, acetic acid, citric acid, lactic acid, acetic acid, ascorbic acid, malic acid, gluconic acid, glutamic acid, hydrochloric acid, sulfuric acid, succinic acid, tartaric acid, or butyric acid.

According to often included embodiments, there is provided a solution for electrospinning as above, wherein the base is sodium hydroxide or potassium hydroxide.

According to frequently included embodiments, there is provided a solution for electrospinning as above, wherein the neutral salt is sodium chloride, potassium chloride, arginine hydrochloride, triethanolamine, or tetraethylammonium bromide.

According to often included embodiments, there is provided a solution for electrospinning as above, wherein the acidic salt is sodium hydrogen sulfate, mono- or di-potassium sulfate, mono- or di-sodium phosphate.

According to frequently included embodiments, there is provided a solution for electrospinning as above, wherein the basic salt is sodium (bi) carbonate or sodium acetate.

According to often included embodiments, there is provided a solution for electrospinning as above, the solution for electrospinning further comprises a wetting agent and/or a penetration enhancer.

According to frequently included embodiments, there is provided a solution for electrospinning as above, wherein the wetting agent or penetration enhancer is anionic surfactants, cationic surfactants, ionic surfactants, fatty acids and derivatives, sulfoxides, alcohols, chelating agents, bile salts and derivatives, cyclodextrins, polyvinylpyrrolidone, lactose, triacetin, and/or menthol.

### BRIEF DESCRIPTION OF THE DRAWINGS

The skilled person in the art will understand that the drawings, described below, are for illustration purposes only.

FIG. 1 shows a view of nanofibrous films with cytomegalovirus glycoprotein B.

FIG. 2 shows a view of Franz's diffusion cell.

FIG. 3 shows the chromatogram for the ovalbumin standard at 214 nm for a concentration of 0.02, 0.05, 0.10, 0.15, and 0.20 mg/ml.

FIG. 4 shows the calibration curve for ovalbumin.

FIG. 5 shows the peak for cytomegalovirus glycoprotein B.

FIG. 6 shows the chromatogram of a sample taken at (a) hour 1 of permeation, (b) hour 2 of permeation, (c) hour 3 of permeation, and (d) hour 4 of permeation.

FIG. 7 shows the permeation profile of cytomegalovirus glycoprotein B (gB) across the porcine sublingual membrane ex-vivo (quantity).

FIG. 8 shows the permeation profile of cytomegalovirus glycoprotein B (gB) across the porcine sublingual membrane ex-vivo (efficacy).

FIG. 9A shows levels of serum IgA antibodies specific to the gB used.

FIG. 9B shows levels of serum IgG antibodies specific to the gB used.

FIG. 10 shows levels of mucosal antibodies specific to the gB used.

FIG. 11A shows levels of IgA antibody-producing cells isolated from the spleens of immunized animals.

FIG. 11B shows levels of IgG antibody-producing cells isolated from the spleens of immunized animals.

FIG. 12 shows cellular memory for Th lymphocytes when using mCMV gB.

FIG. 13 shows a calibration curve between BSA concentration and peak area at 220 nm for films from sample 1.

FIG. 14 shows a calibration curve between BSA concentration and peak area at 220 nm for films from sample 2.

### DETAILED DESCRIPTION

For clarity of disclosure, and not by way of limitation, the detailed description of the invention is divided into the subsections that follow.

Unless defined otherwise, all technical and scientific terms used herein have the same meaning as is commonly understood by one of ordinary skill in the art to which this invention belongs. All patents, applications, published applications, and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

As used herein, “a” or “an” means “at least one” or “one or more.”

As used herein, the term “and/or” may mean “and,” it may mean “or,” it may mean “exclusive-or,” it may mean “one,” it may mean “some, but not all,” it may mean “neither,” and/or it may mean “both.”

As used herein, “oral thin film” or “thin film” refers to a sheet of nanofiber or a portion thereof produced by electrospinning of a polymer solution. Such a sheet may be composed of one or more layers of nanofiber.

As used herein, “saliva permeable” refers to a characteristic of a material wherein saliva in an animal oral cavity can penetrate into or through the material when the material is placed in the animal oral cavity, and/or the material reacts with, breaks down or dissolves in fluids and enzymes in the saliva. For example, a nanofiber matrix material in the form of a thin film may be composed of one or more saliva permeable polymers and the fully formed thin film itself is saliva permeable.

As used herein, “saliva impermeable” refers to a characteristic of a material wherein saliva in an animal oral cavity cannot penetrate into or through the material when the material is placed in the animal oral cavity, and/or the material does not react with, break down or dissolves in fluids and enzymes in the saliva. Often the term “saliva impermeable” is used in the context of a material that maintains the characteristic of saliva impermeability for at least a known period of time, e.g., 1 hour, 2 hours, 3, hours, 4 hours, 5 hours, or longer. For example, a nanofiber matrix material in the form of a thin film may be composed of one or more saliva impermeable polymers and the fully formed thin film itself is saliva impermeable.

Embodiments of this invention relate to a method to vaccinate against cytomegalovirus (CMV) infections using oral thin films embedded with CMV glycoprotein B for oral/oromucosal administration. Subjects receive administration by placing the oral thin films into the subject's oral cavity at different time points according to an administration schedule.

The present disclosure provides pharmaceutical compositions for oromucosal administration, in particular for vaccination against cytomegalovirus infections. According to the present disclosure, one key component of these compositions is cytomegalovirus glycoprotein B as an active ingredient. In a preferred embodiment, the amount of cytomegalovirus glycoprotein B in the pharmaceutical composition is from 2 to 250 μg in a single dose for humans. Vaccine is administered by one (1) prime administration followed by two (2) booster administrations. Additional boosters can be administered thereafter as needed. Other administration schedules are contemplated to fit with the need of particular subjects. Vaccines can be administered as prime administration on day 1, followed by first booster on at least 7 days thereafter, followed by second booster on at least 7 days thereafter. Additional boosters can be administered, at a time gap of at least 7 days after the prior administration.

Cytomegalovirus glycoprotein B is an important antigen that provides both cellular and humoral immune response. The mucosal surface is constantly exposed to numerous pathogens, acting as an entry point for the host body, and in the absence of a mucosal immune response, these pathogens can invade our immune system and cause infections that might result in severe morbidity and/or mortality. In the case of oromucosal administration, the active substance of the pharmaceutical composition, namely cytomegalovirus glycoprotein B, is passed directly to the immune cells located beneath the mucosa in the oral cavity. These immune cells interact with foreign bodies or antigens, such as CMV gB protein, to elicit the desired immune response. The interaction between immune cells and antigen initiates a cascade of immune reactions which leads to the development of both cellular and humoral immune response, and in this case, including mucosal components. Furthermore, in the case of mucosal vaccines with improved accessibility, needle-free delivery, mass immunization, and reduction in overall vaccination cost can be achieved.

In a preferred arrangement, an oral thin film has cytomegalovirus glycoprotein B as the active ingredient. CMV gB is present within the nanofibers of at least one flexible and saliva-permeable active layer, which is covered and/or protected by at least one saliva-impermeable layer, and the active layer and the at least one saliva-impermeable layer comprise the oral thin film. The flexible active layer is moldable to the shape of the selected oral mucosa to which it is to be applied, thereby maximizing the area of contact of the flexible active layer with the selected oral mucosa. This allows maximum permeation of cytomegalovirus glycoprotein B across the oromucosal layer or to immune cells located beneath the oral mucosa. The formability and elasticity of the active layer of the nanofibers are achieved by manufacturing the nanofiber layer using electrospinning technology. The produced nanofibers have enhanced flexibility and surface area into which the active ingredients, in this case, the CMV gB protein is deposited. A polymer solution containing an active ingredient, in this case, CMV glycoprotein B, is subject to a high electric field which initiates the electrospinning process, leading to the formation of nanofibers. The process of production of nanofibers under the influence of a high electric field could be performed as needle-based electrospinning, or methods of needleless electrospinning, such as wired-based electrospinning. During the manufacturing process, glycoprotein B is introduced into the fibers, i.e. it is deposited into the nanofibers of the flexible active layer and is released upon the dissolution of the active layer. Cytomegalovirus glycoprotein B is added into a polymeric solution for needleless electrospinning and the solution is electrospun under the influence of a high electric field. Nanofiber layers produced from this electrospinning process have cytomegalovirus glycoprotein B embedded inside the nanofiber. This arrangement of nanofibers protects the CMV gB protein from any physical or mechanical degradation during processing or handling of the thin film.

When the cytomegalovirus glycoprotein B active agent is deposited, it is deposited within the nanofibers. Coating, dipping, or encapsulation by coaxial and emulsion electrospinning are common methods used to produce nanofibers, nanofiber layers and/or carriers. Electrospinning with a needle can also be used for production of oral films containing cytomegalovirus glycoprotein B. However, it is not feasible to perform industrial-scale production of oral thin films with needle electrospinning. A solution or other dispersion is often used to deposit the active ingredient cytomegalovirus glycoprotein B.

The use of nanofibers produced from a pharmaceutical-compatible composition with a releasably deposited cytomegalovirus glycoprotein B as an active ingredient provides several advantages. The active ingredient can be incorporated into the nanofiber matrix relatively easily and efficiently during the electrospinning or ES process. According to existing knowledge, the intensity of the electric field applied during ES does not have a negative effect on the intrinsic activity of the active substance used, i.e. not even for CMV gB. The large surface area, the elasticity of the nanofibrous layers, and the possibility to choose polymers based on the desired pharmacokinetics gives the nanofibers an added value when compared to the commonly used orodispersion films. The possibility to select a suitable polymer gives the flexibility to modify the release of active substances from the thin film. This ability to control the drug release, from fast to delayed, gives the ability to control the pharmacokinetics of active substances without influencing/changing the structure of the active substance. By using nanofibers, cytomegalovirus glycoprotein B is deposited onto an oromucosal surface. Cytomegalovirus glycoprotein B as the antigen is permeated across the oromucosal membrane and is captured by antigen presenting cells (APC). This interaction between CMV gB and APC initiates the cascade of immune reactions leading to the antigen specific cellular and humoral immune response. In principle, the mode of action for antigen is different as compared to other active pharmaceutical ingredients. In the former case, the uptake of active substance is through APC, and in the latter case, the active substance enters the bloodstream to initiate the relevant physiological response. During electrospinning process, the native structure of cytomegalovirus glycoprotein B and its functionality is not affected, and it remains embedded within the nanofibers. The structural property of nanofibers and the pharmaceutical composition of the film protects cytomegalovirus glycoprotein B not only during storage, not requiring adherence to a strict cold chain of distribution, but also protects it after the actual application from environmental influences in the oral cavity. The covering/protective layer protects the CMV gB from enzymatic degradation in the oral cavity, leaching with saliva and passing through gastrointestinal tract. This maximizes the amount of the substance that remains in the active form at the site of application.

The use of pharmaceutically acceptable polymers and/or derivatives thereof is generally preferred for the preparation of the flexible active layer and the carrier cover/protective layer of the pharmaceutical composition. Polymer derivatives are polymers with different molecular weights or functional group modifications, including copolymers, cross-linked polymers, or combinations thereof. Such polymers may be commercially available or may be prepared using known techniques.

The flexible active layer and the covering/protective layer, or solutions used to prepare such layers, may comprise a mixture of polymers, including water-soluble or hydrophilic polymers and water-insoluble or hydrophobic polymers or biodegradable polymers, together in a layer or solution.

Hydrophilic or water-soluble polymers include, for example, some of the following polymers including their derivatives and salts: polyethylene glycols (PEG), polyethylene oxides (PEO), copolymers of polyethylene oxide with other alkylene oxides, in particular as block copolymers of PEG and polypropylene oxide or polybutylene oxide, polyvinyl alcohol, polyvinylpyrrolidones (PVP), albumin, dextran, hyaluronic acid, alginates, carrageenan, chitosan, gelatin, collagen, zein and derivatives, polyacrylic acid, cellulose derivatives such as sodium carboxymethyl cellulose, hydroxyethyl cellulose (HEC), hydroxypropyl cellulose (HPC), hydroxypropyl methylcellulose (HPMC), hydroxypropyl methylcellulose phthalate, cellulose acetate phthalate, oxidized cellulose (e.g. Cellulose, cellulose acetate, cellulose in micro-dispersed form). Hydrophilic polymers are generally water-soluble polymers.

Biodegradable polymers may include certain hydrophilic or hydrophobic polymers but often have certain hydrophobic properties. Some exemplary biodegradable polymers include polycaprolactone, polylactic acid, poly(lactide-co-glycolide), polyglycolic acid.

Polymers used for needleless electrospinning to produce the active layer and/or the coating layer include, for example, polycaprolactone (PCL), polyvinyl alcohol (PVA), polyacrylic acid (PAA).

Hydrophobic polymers include, for example, some of the following polymers including their derivatives and salts: Cellulose derivatives (acetylcellulose, methylcellulose, ethylcellulose, noncrystalline cellulose); polymers based on (meth)acrylates (i.e., acrylates, methacrylates, alkylacrylates and copolymers thereof), including (meth)acrylic acid, (meth)acrylamides, hydroxyethyl (meth)acrylates; poly(alpha-hydroxy acids) and its copolymers such as poly(c-caprolactone), poly(lactide-co-glycolide), poly(alpha-aminoacids) and its copolymers; polyurethanes (all with approval for usage in medical/pharmaceutical field). In certain embodiments, hydrophilic polymers are treated to have hydrophobic properties and/or become water-insoluble or partially water-insoluble, for example when utilized in the coating layer.

The flexible active layer may comprise a wetting agent and/or a penetration enhancer such as: an anionic surfactant, e.g., sodium laurate, sodium lauryl sulfate, sodium dodecyl sulfate, sodium dioctyl sulfosuccinate, sodium glycocholate, ionic surfactants, e.g., polysorbates, nonyl phenoxy poly oxyethylene, polyoxyalkylene, polyoxyethylene alkyl derivatives, or cationic surfactants, e.g. cetylpyridinium chloride, poly-L-arginine; fatty acids and derivatives such as oleic acid, lauric acid, linoleic acid, acetylcholine, acylcarnitine, monoglycerides, diglycerides and triglycerides and/or caprylic acid; sulfoxides such as dimethyl sulfoxide and/or dodecyl sulfoxide; alcohols including ethanol, isopropanol, propylene glycol, glycerol, propanediol and/or menthol; chelating agents such as EDTA, citric acid and/or salicylates; bile salts and derivatives and other compounds such as cyclodextrins, polyvinylpyrrolidone, lactose, triacetin and/or menthol. In certain embodiments, the hydrophilic polymers are modified to have hydrophobic properties and/or to become water-insoluble or partially water-insoluble. In certain embodiments, the coating layer may also comprise a wetting agent and/or penetration agent, as above. In certain embodiments, there could be an additional layer with wetting agent or penetration enhancer.

To facilitate the electrospinning process, conductive agents helping to achieve the desired conductivity of an electrospinning solution are used. Conductive agents include certain acids, such as phosphoric acid, formic acid, acetic acid, citric acid, lactic acid, acetic acid, ascorbic acid, malic acid, gluconic acid, glutamic acid, hydrochloric acid, sulfuric acid, succinic acid, tartaric acid, butyric acid, arginine hydrochloride; certain bases, such as sodium hydroxide, potassium hydroxide; neutral salts, such as sodium chloride, potassium chloride, arginine hydrochloride, triethanolamine, tetraethylammonium bromide; acidic salts, such as sodium hydrogen sulfate, mono- or di-potassium sulfate, mono- or di-sodium phosphate; or basic salts, such as sodium (bi) carbonate or sodium acetate. Other agents capable of dissociating to create a charged environment conducive to electrospinning are contemplated.

The pharmaceutical composition comprising cytomegalovirus glycoprotein B according to the present invention is for use in the prevention or treatment of cytomegalovirus infections.

It is also an object of the invention to use cytomegalovirus glycoprotein B to produce a vaccine for oromucosal vaccination against cytomegalovirus infections.

With respect to ex-vivo conditions, proteins are more susceptible to degradation compared to in-vivo sublingual administration. Cytomegalovirus glycoprotein B is more protected when embedded in nanofibers within films produced according to embodiments herein. The solution according to the present invention promotes sublingual administration of cytomegalovirus glycoprotein B and its release from the carrier in the form of a nanofibrous film. The advantages of the pharmaceutical composition for oromucosal administration, in particular for vaccination against cytomegalovirus infections, according to the present invention are that it exhibits higher bioavailability compared to other oral vaccines, stimulates mucosal immunity, allows targeting and fixation of the vaccine agent to the sites inducing the immune cascade in the mucosa. The pharmaceutical composition according to embodiments herein sufficiently stimulates the immune system so that it generates effective adaptive immunity without side effects, thus preventing infection or its reactivation. The presented embodiments and the mode of administration reduce the dependence on oral administration of virostatics and associated side effects.

### Examples

### Preparation of Pharmaceutical Composition

Recombinant cytomegalovirus glycoprotein B or CMV gB (UL55) from MyBioSource (CA, USA) was used to prepare a pharmaceutical composition according to the present invention.

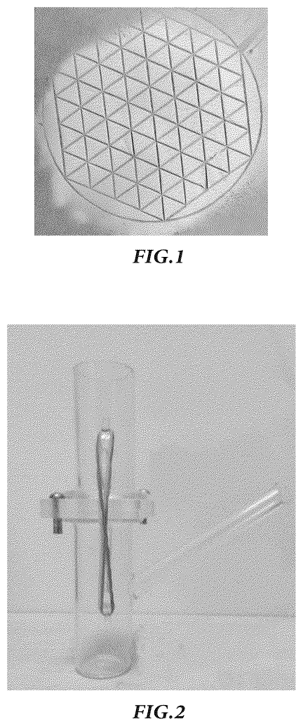

Hydroxypropyl methylcellulose (HPMC), polymer polyvinyl alcohol or PVA, and polyethylene oxide (PEO) were mixed with cytomegalovirus glycoprotein B or CMV gB. The mixture thus prepared was used to prepare a pharmaceutical composition in the form of nanofibrous film. The preparation was carried out on a Nanospider™ NS 1WS500U apparatus by electrostatic wetting technology. The nanofibrous film layer was cut into triangular films with each side dimension 5 mm using the GCC Mercury III laser system as shown in FIG. 1. The triangular nanofibrous film contained 10±0.1 μg of cytomegalovirus glycoprotein B.

The flexible active layer with CMV gB was prepared by electrospinning technology using the following parameters: wire electrode distance 180 mm; relative humidity in the machine between 27 and 31% and temperature between 21 and 24.5° C.; voltage 60/−18 kV. In such an embodiment, medical paper is provided as the layer deposition material. The coating layer was produced by the same electrospinning process using the following exemplary parameters: wire electrode distance 195 mm; relative humidity in the machine between 27 and 31% and temperature between 21 and 24.5° C.; voltage 60/−28 kV.

The pharmaceutical composition thus prepared contains:

The carrier coating of the pharmaceutical composition is formed by a mixture:

HPC, PVA, and PEO were dissolved in water to form individual polymer solutions, then mixed together. By way of an example, HPC polymer solution is at about 7% to 15% w/w concentration, PVA polymer solution is at 8% to 12% w/w concentration, and PEO polymer solution is at 2% to 8% w/w concentration. Other ingredients are then added.
(a) calculated as % w/w in the prepared solution:
HPC polymer solution 41.02%
PVA polymer solution 33.80%
PEO polymer solution 15.79%
Opadry KB (Coloring agent) 0.94%
KOH (conducting agent) 0.34%
Glycerol 0.60%
Water 7.51%

The prepared solution is subject to needle or needleless electrospinning to produce a coating layer.

The flexible active layer of the carrier containing cytomegalovirus glycoprotein B is composed of a polymer mixture containing HPMC, PVA, and PEO. At first, HPMC was dissolved in water to form a polymer solution, with a concentration of 11% to 24%. PVA was dissolved in water to form a polymer solution with a concentration of 8% to 12% (w/w) and PEO was dissolved into water to form a polymer solution with a concentration of 2% to 8% (w/w). Later, HPMC, PVA, and PEO were mixed together with sodium chloride, water, glycerol, then the active agent, CMV glycoprotein B, was added together with glycerol under constant stirring.
(a) calculated as % w/w in the prepared solution:
HPMC solution 15.54%
PVA solution 20.71%
PEO solution 6.27%
Active agent CMV gB 0.26%
Water 42.70%
Glycerol 14.52%

The prepared solution is subject to needle or needleless electrospinning to produce a flexible active layer.

A coating layer is produced by electrospinning by depositing the nanofiber onto a receiving mat. An active layer is then produced by electrospinning by depositing onto the coating layer previously produced. An additional layer may be produced by electrospinning by depositing onto the active layer previously produced. The additional layer is optional and can be another coating layer or another active layer.

Films produced are stable at 2-8° C. and are stored in primary packaging materials comprised of aluminum and polyethylene materials.

### Measurement of Protein Content in Strips Loaded with CMV gB

### CMV Films

### 1) CMV Sample 1:

Active substance used for the films: Cytomegalovirus glycoprotein B (UL55) (CMV gB), Recombinant Protein (purity >90% as determined by SDS-PAGE). Final product: triangular film with each side of dimension 5 mm, white color, without spots.

### 2) CMV Sample 2:

Active substance used for films: Human cytomegalovirus (HCMV) Glycoprotein B/gB Protein (purity >70% as determined by SDS-PAGE). Final product: triangular film with each side of dimension 5 mm, white color, without spots.

### Standard

| | Albumin Standard |
|---|---|
| Standard | LOT: VG298380 |
| Standard for system | standard BSA_STD_100% (0.02 mg/ml) |
| proficiency test | |
| Mobile phase | Water purified, bag in box 5l |
| | batch no.: 22F27-T19-097804 |
| | Phosphate buffered saline (PBS) tbl. |
| | Batch no.: SLCJ5627 |

| | |
|---|---|
| Liquid | Agilent 1260, UV/VIS a FLD detektors (Agilent) |
| chromatograph | SOP-QC-801 |
| Automatic | Finnpipette 1-10 ml, 100-1000 μl, 20-200 μl |
| micropipetes | SOP-QC-803 |
| Mobile phase | Piston vacuum pump (without oil) |
| filtration | SOP-QC-815 |
| Mobile phase | Ultrasonic bath |
| degassing | SOP-QC-805 |

The content analysis of CMV gB was performed using the following HPLC parameters:
Mobile phase: PBS (pH:7.2-7.5)
Guard: without
Column: without (Column bypass)
Column temperature: 25° C.
Flow: 0.5 ml/min
Analysis length: 2 min
Injection: 5.0 μl
UV detection: 220 nm
Retention time (RT) peak protein: approx. 0.1 min

### Sample Preparation

Sample 1: Nanofiber layer loaded with Human cytomegalovirus (HCMV) Glycoprotein B protein (Sino Biological Inc.) was produced. The layer was cut into triangular film (strip) with each side of dimension 5 mm.

CMV gB Strip: Each strip was dissolved in 300 μl PBS.

BSA (albumin from bovine serum) Standards: The standard concentration is expressed in the concentration of proteins in mg/ml.

### Calibration Curve:

| BSA Standard | Conc (mg/mL) |
|---|---|
| Blank | 0.000 |
| SBSA 0.02 | 0.020 |
| SBSA 0.04 | 0.040 |
| SBSA 0.06 | 0.060 |
| SBSA 0.08 | 0.080 |
| SBSA 0.10 | 0.100 |
| SBSA 0.12 | 0.120 |
| SBSA 0.14 | 0.140 |

Sample 2: Nanofiber layer loaded with Cytomegalovirus glycoprotein B (gB) (UL55) recombinant protein (MyBioSource Inc.) was produced. The layer was cut into triangular film (strip) with each side of dimension 5 mm. As the CMV gB protein source from MyBioSource included 10 mM Glutathione, a placebo strip loaded with 10 mM Glutathione was included in the study.

| | Strip | Expected |
|---|---|---|
| CMV | weight | CMV gB |
| Strip | (mg) | content (μg) |
| Placebo | — | 0 |
| 1 | 0.44 | 5.76 |
| 2 | 0.43 | 5.63 |
| 3 | 0.46 | 6.03 |
| 4 | 0.44 | 5.76 |
| 5 | 0.40 | 5.24 |

CMV gB Strip: Each strip was dissolved in 250 μl PBS.

BSA Standards: The standard concentration is expressed in the concentration of proteins in mg/ml.

### Calibration Curve:

| BSA Standard | Conc (mg/mL) |
|---|---|
| Blank | 0.000 |
| BSA_STD_50% | 0.010 |
| BSA_STD_80% | 0.016 |
| BSA_STD_100% | 0.020 |
| BSA_STD_120% | 0.024 |
| BSA_STD_140% | 0.028 |

### System Proficiency Test

6 injections from the same standard sample BSA_STD_100% were analyzed to verify the repeatability of injections, at the beginning and end of the sequence

Table 1 demonstrates the repeatability of the method in terms of peak area, SD, and RSD.

TABLE 1
System Proficiency Test

| | Line | Sample | RT [min] | Area |
|---|---|---|---|---|
| Start | 3 | BSA_STD_100%-31.10. | 0.117 | 965.9 |
| | 4 | BSA_STD_100%-31.10. | 0.117 | 964.1 |
| | 5 | BSA_STD_100%-31.10. | 0.118 | 963.2 |
| | 6 | BSA_STD_100%-31.10. | 0.117 | 961.0 |
| | 7 | BSA_STD_100%-31.10. | 0.117 | 962.8 |
| | 8 | BSA_STD_100%-31.10. | 0.117 | 962.4 |
| End | 37 | BSA_STD_100%-31.10. | 0.117 | 958.8 |
| | 38 | BSA_STD_100%-31.10. | 0.117 | 958.6 |
| | 39 | BSA_STD_100%-31.10. | 0.117 | 960.3 |
| | 40 | BSA_STD_100%-31.10. | 0.118 | 959.3 |
| | 41 | BSA_STD_100%-31.10. | 0.117 | 955.9 |
| | 42 | BSA_STD_100%-31.10. | 0.117 | 957.0 |

| Start | | End | |
|---|---|---|---|
| Mean | 963.233 | Mean | 958.317 |
| SD | 1.657 | SD | 1.599 |
| RSD | 0.172% | RSD | 0.167% |

Acceptability criterion: RSD <2%
Conclusion: The results obtained were in accordance with the acceptability criteria.

### Analysis Results:

### Content

The CMV gB content in each film was calculated using the BSA standard. Depending on the expected CMV gB content in films, BSA standard points for the calibration curve were determined. In the case of films from sample 1, BSA standard in the range of 0.020-0.140 mg/mL was used. Further, the concentration of BSA and respective peak area at 220 nm was used to formulate the calibration curve which was then used to calculate the CMV gB content in films. In the case of films from sample 2, BSA standard in the range of 0.010-0.028 mg/mL was used. BSA Standard 100% represents the ideal concentration expected to be achieved upon dissolution of film loaded with CMV gB. A calibration curve is designed to include the entire range of possible concentrations or amounts of protein expected to be present in a sample. The film was designed to achieve the loading target of 5 μg/film, and 100% represents the concentration when the film with the loading of 5 μg is dissolved in 250 μl of phosphate buffer to result in the solution of 0.02 mg/ml. Further, the concentration of BSA and respective peak area at 220 nm was used to formulate the calibration curve which was then used to calculate the CMV gB content in films. In both cases, the amount of CMV gB in each film was extrapolated from the calculated concentration by considering the volume of phosphate buffer used to dissolve the film.

### Sample No. 1:

Standard BSA: Table 2 - BSA Standard: Absorbance at 220 nm

| | | Absorbance in | | | |
|---|---|---|---|---|---|
| | Conc | duplicates | | | Without |
| S. No. | (mg/mL) | Value 1 | Value 2 | Avg. | Blank |
| Blank | 0.000 | 47.9 | 53.9 | 50.9 | 0 |
| 1 | 0.020 | 918.2 | 920.5 | 919.35 | 868.45 |
| 2 | 0.040 | 1860.6 | 1859.7 | 1860.15 | 1809.25 |
| 3 | 0.060 | 2872.7 | 2875.6 | 2874.15 | 2823.25 |
| 4 | 0.080 | 3991.2 | 3988.1 | 3989.65 | 3938.75 |
| 5 | 0.100 | 4942 | 4950.1 | 4946.05 | 4895.15 |
| 6 | 0.120 | 5972.7 | 5955.4 | 5964.05 | 5913.15 |
| 7 | 0.140 | 6984.5 | 6973.2 | 6978.85 | 6927.95 |

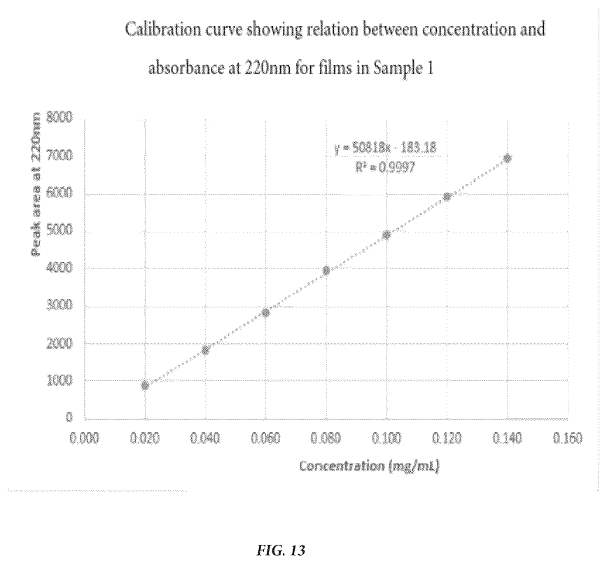

A plot of a calibration curve between BSA concentration and peak area at 220 nm for films from sample 1 is provided in FIG. 13.

### Table 3

TABLE 3
CMV gB content in each film

| | | CMV | | | | | |
|---|---|---|---|---|---|---|---|
| | | gB | | | | | |
| | | Loading | | | | | CMV gB |
| | Strip | (μg) - | Absorbance at 220 | | | | (μg) - |
| CMV | weight | Theoretical | nm in duplicates | | | Conc. | Calculated |
| gB Film | (mg) | value | Value 1 | Value 2 | Avg. | (mg/mL) | value |
| 1 | 2.07 | 9.94 | 1466.6 | 1409.4 | 1438.00 | 0.062 | 9.270 |
| 2 | 1.84 | 8.83 | 1373.7 | 1370.2 | 1371.95 | 0.059 | 8.880 |
| 3 | 2.11 | 10.13 | 1584 | 1582 | 1583.00 | 0.068 | 10.126 |
| 4 | 1.98 | 9.50 | 1505.6 | 1506.2 | 1505.90 | 0.064 | 9.671 |
| 5 | 1.9 | 9.12 | 1370.3 | 1339 | 1354.65 | 0.059 | 8.778 |
| 6 | 2.03 | 9.74 | 1487.3 | 1520.3 | 1503.80 | 0.064 | 9.658 |
| 7 | 2.06 | 9.89 | 1384.8 | 1447.3 | 1416.05 | 0.061 | 9.140 |
| 8 | 1.92 | 9.22 | 1459.6 | 1473.2 | 1466.40 | 0.063 | 9.438 |
| 9 | 2.17 | 10.42 | 1757.5 | 1741.5 | 1749.50 | 0.074 | 11.109 |
| 10 | 2.05 | 9.84 | 1230.7 | 1296.6 | 1263.65 | 0.055 | 8.241 |

### Sample No. 2:

TABLE 4
Standard BSA: BSA Standard: Absorbance at 220 nm

| | | | Absorbance at 220 | | | |
|---|---|---|---|---|---|---|
| | | Conc | nm in duplicates | | | Without |
| S. No. | Content | (mg/mL) | Value 1 | Value 2 | Avg. | Blank |
| Blank | 0% | 0.000 | 42.3 | 42.5 | 42.4 | 0 |
| 1 | 50% | 0.010 | 577.4 | 577.5 | 577.45 | 535.05 |
| 2 | 80% | 0.016 | 840.7 | 839.9 | 840.3 | 797.9 |
| 3 | 100% | 0.020 | 961.7 | 964.2 | 962.95 | 920.55 |
| 4 | 120% | 0.024 | 1075.4 | 1075.4 | 1075.4 | 1033 |
| 5 | 140% | 0.028 | 1211.5 | 1228.3 | 1219.9 | 1177.5 |

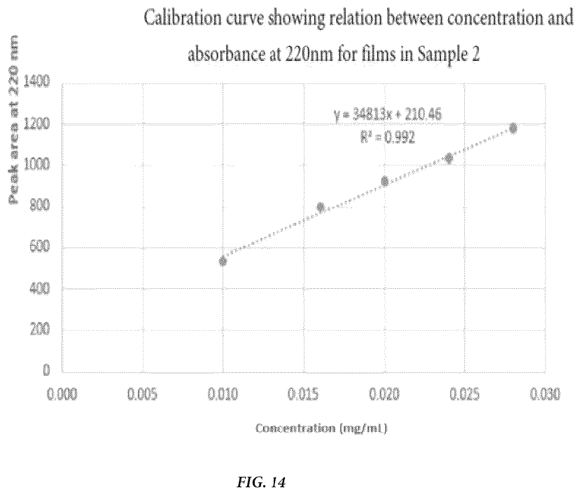

A plot of a calibration curve between BSA concentration and peak area at 220 nm for films from sample 2 is provided in FIG. 14.

TABLE 5
CMV gB content in each film

| | | CMV gB | | | | | |
|---|---|---|---|---|---|---|---|
| | | Loading | | | | | CMV |
| | Strip | (μg) - | Absorbance at 220 | | | | gB (μg) - |
| CMV | weight | Theoretical | nm in duplicates | | | Conc. | Calculated |
| gB Film | (mg) | value | Value 1 | Value 2 | Avg. | (mg/mL) | value |
| Placebo | — | — | 1129.3 | 1127.7 | 1128.5 | −0.006 | 0 |
| 1 | 0.44 | 5.76 | 2091 | 2086.2 | 2088.6 | 0.022 | 5.383 |
| 2 | 0.43 | 5.63 | 2160.1 | 2182.5 | 2171.3 | 0.024 | 5.977 |
| 3 | 0.46 | 6.03 | 2354.7 | 2365.2 | 2359.95 | 0.029 | 7.332 |
| 4 | 0.44 | 5.76 | 2011.3 | 2000.8 | 2006.05 | 0.019 | 4.791 |
| 5 | 0.4 | 5.24 | 2214.5 | 2213.4 | 2213.95 | 0.025 | 6.284 |

Conclusion: The estimated CMV gB content in the films of sample 1 and sample 2 were in the expected range.

### Design of the CMV gB Permeation Experiment

To monitor the permeation of cytomegalovirus glycoprotein B across the mucosa, a Franz diffusion cell was set up using a flat flange joint and silicone gel as shown in FIG. 2. The sublingual or sublingual membrane was extracted from a fresh porcine tongue and was attached between the flat flange joint using silicone gel. The donor buffer was prepared by dissolving 9.52 g KH₂PO₄ and 0.68 g NaCl in 950 ml of purified water. The pH of the donor buffer was adjusted to 6.8 using 1M NaOH, to mimic the pH of saliva, and the final volume was adjusted to 1000 ml. The acceptor buffer was prepared by dissolving 1.18 g Na₂HPO₄·2H₂O, 0.19 g KH₂PO₄, 8.0 g NaCl in 950 ml of purified water. The pH of the acceptor buffer was adjusted to 7.4 with 1M NaOH, to mimic the pH of blood, and the final volume was adjusted to 1000 ml.

A triangle-shaped nanofibrous film containing 3.5±0.35 μg of cytomegalovirus glycoprotein B was placed on a sublingual membrane adhered between the flat flange junction of the Franz diffusion cell and immersed in donor buffer, which is at the top of the Franz diffusion cell. The flat flange joint on the donor side was covered with a cover slip to prevent loss of the donor buffer during incubation at 25° C. Six Franz diffusion cell devices were set up, two with a nanofibrous film loaded with cytomegalovirus glycoprotein B, two with a placebo nanofibrous film, and two without any film as a control. Acceptor buffer was added to the bottom of the Franz diffusion cell after assembly using a syringe. The presence of any air bubbles in the acceptor buffer was carefully avoided, as this could affect the efficiency of the permeation process. In each case, the volume of donor and acceptor buffer added was recorded as shown in Table 6 below:

TABLE 6

| Franz | Active | Donor Buffer | Acceptor buffer |
|---|---|---|---|
| diffusion cell | substances | (pH 6.8) | (pH 7.4) |
| FC 1 | CMV gB protein | 0.1 ml | 18.5 ml |
| FC2 | CMV gB protein | 0.1 ml | 18.5 ml |
| FC 3 | Placebo | 0.1 ml | 15.8 ml |
| FC4 | Placebo | 0.1 ml | 16 ml |
| FC5 | check | 0.1 ml | 16 ml |
| FC6 | check | 0.1 ml | 17.8 ml |

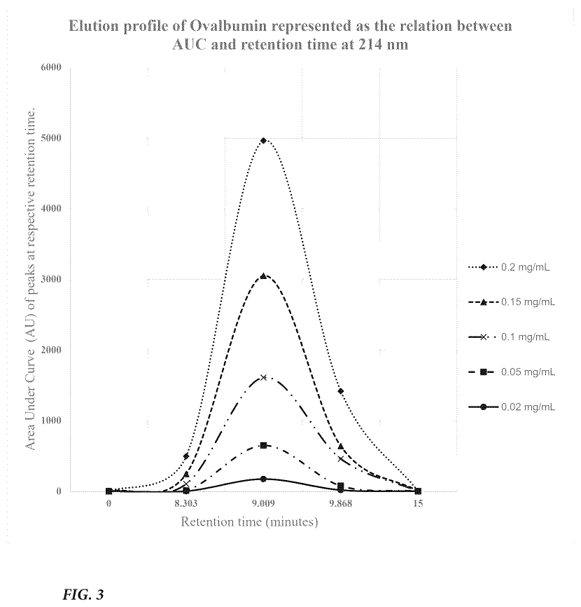

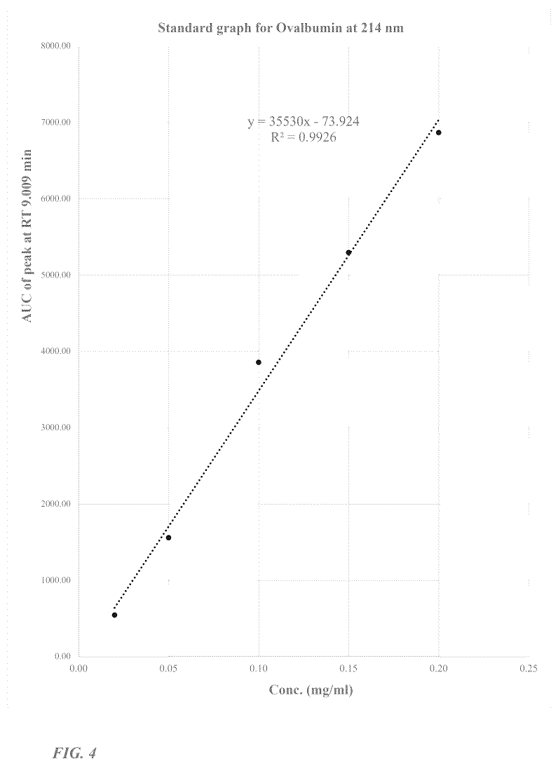

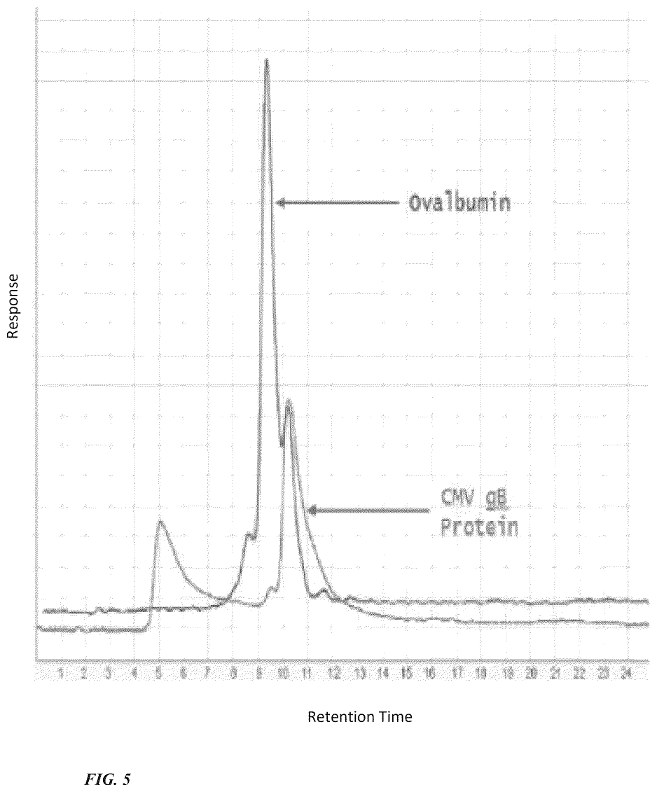

As the molecular weight of cytomegalovirus glycoprotein B used for this study (39 kDa) was close to that of ovalbumin (45 kDa), ovalbumin was used as the standard for the calibration curve. A 1 mg/ml ovalbumin standard was prepared by dissolving 10 mg of ovalbumin (Sigma Aldrich) in 10 ml of acceptor buffer. A five-point calibration curve was generated by diluting the standard to 0.02, 0.05, 0.10, 0.15, and 0.20 mg/ml as shown in FIG. 3. To identify the peak representing cytomegalovirus glycoprotein B (CMV gB standard), 4 μg of CMV gB protein was dissolved in 0.2 ml of acceptor buffer and analyzed using HPLC. Ovalbumin was analyzed at 214 nm and a calibration curve between peak area and concentration was plotted as shown in FIG. 4.

The assembled Franz diffusion cell with acceptor and donor buffers was incubated at 25° C. using a water bath. Sampling was performed from each cell at predetermined time points through the sampling port as shown in Table 7 below. At each time point, 1 ml of acceptor buffer was removed from the sample cells and supplemented with 1 ml of fresh acceptor buffer. After completion of the incubation period of 4 h, each collected sample was filtered using a 0.2 um polytetrafluoroethylene or PTFE filter and stored at 4° C. until analysis.

TABLE 7

Incubation time

| | Start | 1 hour | 2 hours | 3 hours | 4 hours |
|---|---|---|---|---|---|
| FC 1 | 10:55 | 11:55 | 12:55 | 13:55 | 14:55 |
| FC 2 | 10:59 | 11:59 | 12:59 | 13:59 | 14:59 |
| FC 3 | 11:24 | 12:24 | 13:24 | 14:24 | 15:24 |
| FC4 | 11:32 | 12:32 | 13:32 | 14:32 | 15:32 |
| FC 5 | 11:34 | 12:34 | 13:34 | 14:34 | 15:34 |
| FC 6 | 11:40 | 12:40 | 13:40 | 14:40 | 15:40 |

All samples including the ovalbumin standard were analyzed using an Agilent 1260 Infinity LC installed with an Agilent SEC-5 column, 5 μm, 300 A, 4.6×150 mm. Isocratic elution was performed using 132 mM KH₂PO₄, pH 7, as the mobile phase at a flow rate of 0.35 ml/min for 25 min. Detection was performed at 214 nm.

A Franz diffusion cell without any nanofibrous film was considered as a control for the determination of proteins native to the porcine sublingual membrane, which could have diffused into the acceptor buffer. It was also used as a reference for the identification of the peak associated with the proteins naturally occurring on the porcine sublingual membrane. These reference peaks were used to eliminate non-specific proteins in the case of samples with a nanofibrous film containing cytomegalovirus glycoprotein B.

The retention time of the peak in the case of 4 μg cytomegalovirus glycoprotein B dissolved in 0.2 ml of buffer (CMV gB Standard) was used as a reference for peak identification in the case of samples collected from the Franz diffusion cell, as shown in FIG. 5. The chromatogram for the CMV gB Standard showed two peaks, the peak at a retention time of ~10.23 min was identified as representing cytomegalovirus glycoprotein B. The identification was also based on the retention time of ovalbumin, the right shift of the cytomegalovirus glycoprotein B peak compared to ovalbumin contributes to its lower molecular weight (39 kDa) compared to ovalbumin (45 kDa).

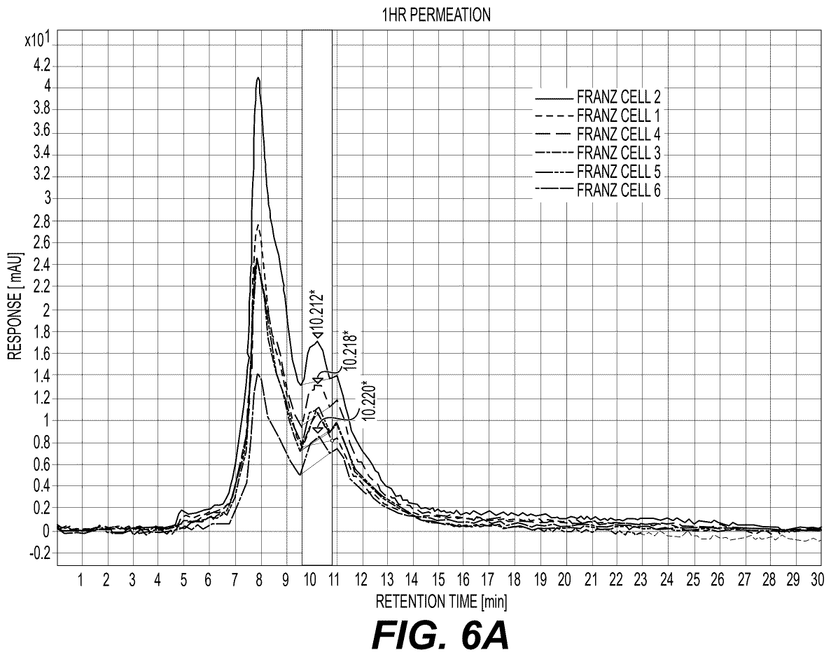

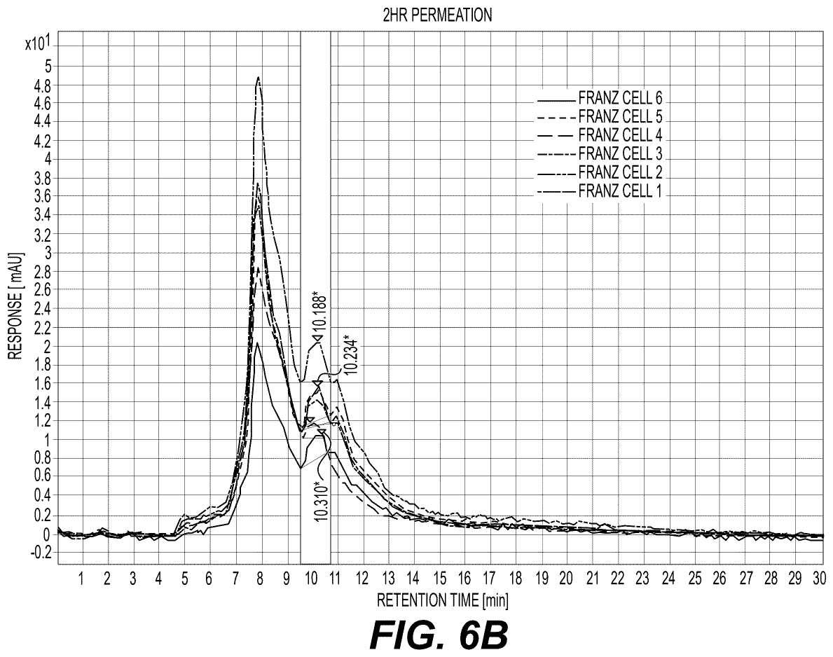

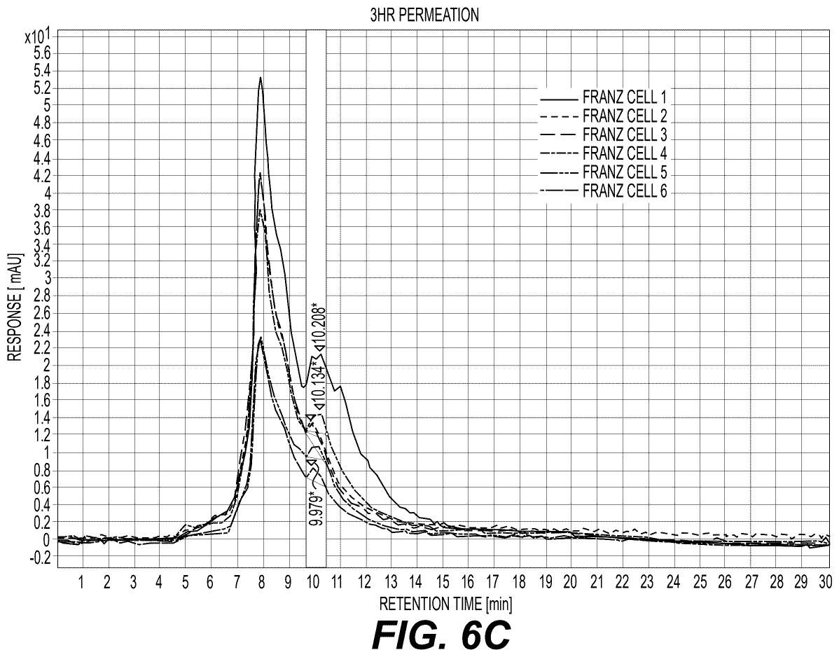

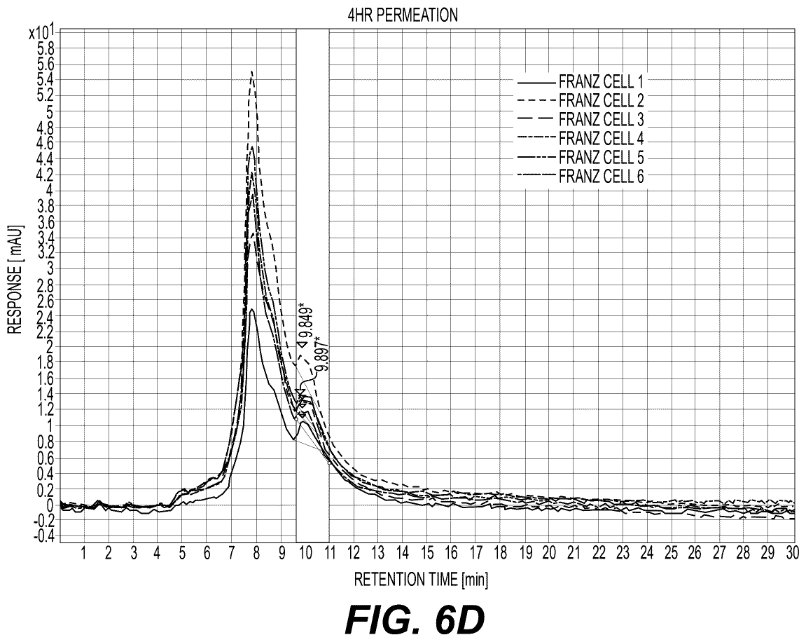

However, in the case of the samples after 3 h and 4 h, the peak was shifted from a retention time of ~10.23 min to ~9.90 min, as shown in FIG. 6. This could be due to some possible interaction between cytomegalovirus glycoprotein B and a protein naturally occurring on the pig tongue.

To calculate the amount of cytomegalovirus glycoprotein B permeated or passed across the porcine sublingual membrane, the peak area in the case of the control was subtracted from the chromatogram of the sample. The final peak area thus obtained was used to calculate the cytomegalovirus glycoprotein B permeated across the sublingual membrane as shown in FIG. 7. The amounts of gB protein permeated across ex-vivo porcine sublingual membrane in 4 hours are shown in Table 8.

TABLE 8

| | Loading | CMV gB Protein Permeated | | | |
|---|---|---|---|---|---|
| Sample | (μg) | 1 hour | 2 hour | 3 hour | 4 hour |
| FC 1 | 3.537 | 0.000 | 0.878 | 0.473 | 0.000 |
| FC 2 | 3.668 | 1.098 | 1.287 | 1.800 | 0.223 |
| Placebo 1 | — | 0.000 | 0.416 | 0.000 | 0.109 |
| Placebo 2 | — | 0.181 | 0.000 | 0.000 | 0.000 |
| Blank 1 | — | 0.054 | 0.000 | 0.022 | 0.780 |
| Blank 2 | — | 0.000 | 0.000 | 0.000 | 0.000 |

The extent of CMV gB protein permeated from oral films across ex-vivo porcine sublingual membrane is shown in Table 9.

TABLE 9

| | Film | | | | | |
|---|---|---|---|---|---|---|
| | Wt. | Loading | Permeation Percentage | | | |
| Sample | (mg) | (μg) | 1 hour | 2 hour | 3 hour | 4 hour |
| FC 1 | 0.27 | 3.537 | 0.00% | 24.83% | 13.37% | 0.00% |
| FC 2 | 0.28 | 3.668 | 29.93% | 35.09% | 49.07% | 6.08% |

In the case of FC 1, total cytomegalovirus glycoprotein B permeated at the end of the third hour was observed to be lower than at the end of the second hour, and no cytomegalovirus glycoprotein B was observed at the fourth hour. And for FC 2, the total permeation for cytomegalovirus glycoprotein B significantly decreased at the fourth hour. These findings may be due to thermal degradation of the protein at 25° C. in the fourth hour of permeation.

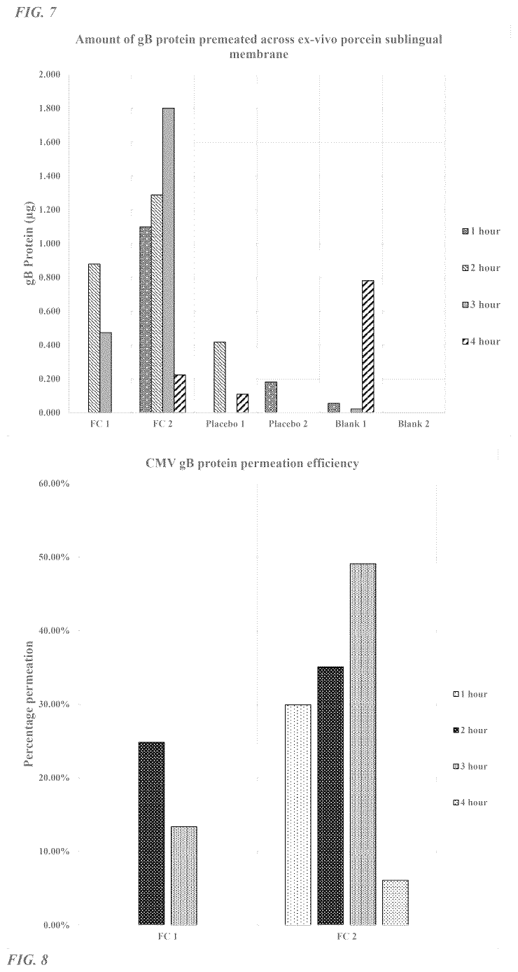

Finally, the efficacy of cytomegalovirus glycoprotein B permeated across the sublingual membrane when applied using a nanofibrous film was evaluated based on the extent of permeation of cytomegalovirus glycoprotein B, as shown in FIG. 8. It can be observed that the second hour can be considered as the absolute time point for permeation of more than 20% of the active substance, i.e. cytomegalovirus glycoprotein B, as shown in FIG. 7. The differences in the permeation values in the samples in Franz diffusion cells may be due to the fact that the extraction of the sublingual membrane from the porcine tongue is most likely non-uniform, leading to non-uniform permeation of the active substance.

### Immune Response Study

Immune response in mouse model administered with the pharmaceutical composition for oromucosal administration according to embodiments herein is studied. The study was performed to evaluate the immunogenic response in mice when CMV gB protein is administered sublingually as a film and compare the response with the intramuscular administration. A negative control group, or mice receiving no immunization, is used for control purposes. Vaccination of the mouse model took place according to the prime and 2× booster scheme. Responses in both the humoral and cellular components of the immune response were monitored. Additional boosters can be administered as needed.

Oral thin films for sublingual administration were produced as a two-layered film with InStrip Technology, which is produced by wire-based electrospinning of a polymer solution. First, the coating or protective layer was produced, followed by the active layer containing an active ingredient, in this case CMV gB. The production was carried out in two batches, for each batch CMV gB protein of different variants was used. For the first batch murine CMV (mCMV) gB protein (39 kDa) (MyBioSource) was used, whereas for the second batch human CMV (hCMV) gB protein (93.2 kDa) (SinoBiologicals) was used. The production of both batches was carried out under the same conditions and the content of the film for both batches was kept the same. Each film was loaded with ~10 μg of CMV gB protein and cellulose polymers.

The in-vivo mice study was performed as a prime and 2 times booster scheme. The mice under study were categorized into three (3) groups, Groups 1 and 2 included six (6) mice for each group, and Group 3 included four (4) mice. Group 1 mice were administered with InStrip oral thin films and were labeled as “nano_gB”, Group 2 mice underwent intramuscular administration and were labeled as “gB_IM”, and Group 3 mice were designated as the negative control and were labeled as “NK”. For each dosing, both prime and booster, each mouse was administered 10 μg of the antigen. Group 1 mice were administered sublingually with oral thin films. Group 2 mice were administered with intramuscular injections. In the case of intramuscular injection, antigens identical to those present in oral thin films were used. The first and second boosters were on the 14^{th} and 28^{th} day, respectively, from the day of priming, which was followed by collecting blood, samples, animal sacrifice, and collecting spleen on the 42^{nd} day. For the negative control group, no administration was performed.

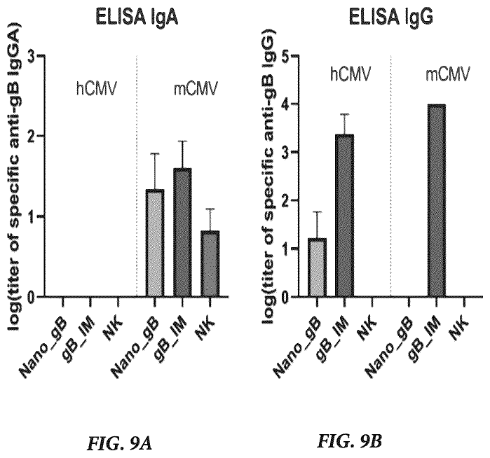

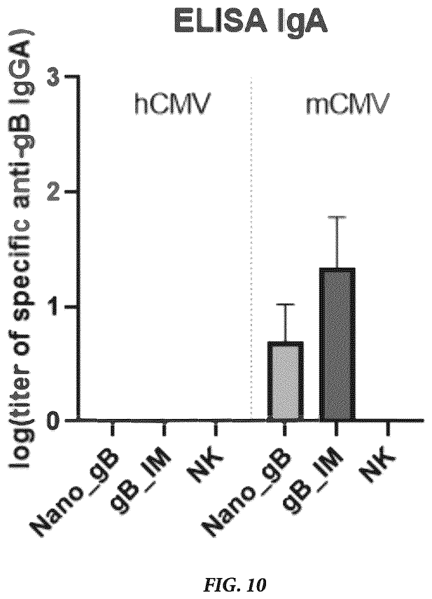

FIGS. 9A & 9B shows the resulting levels of serum Immunoglobulin A (IgA) and Immunoglobulin G (IgG) antibodies specific to the gB used. FIG. 10 illustrates the obtained levels of mucosal IgA determined from vaginal lavage. The results show that both used gB variants were able to induce a humoral response in the tested animals. The human gB variant (hCMV) induced a response only in IgG antibody titers in the Nano-gB and gB_IM immunized groups. The murine variant (mCMV) then stimulated the production of both IgA and IgG antibody classes. A mucosal response was noted only when mCMV gB was used. The increase in specific IgA was confirmed in both the Nano_gB and gB_IM animal groups.

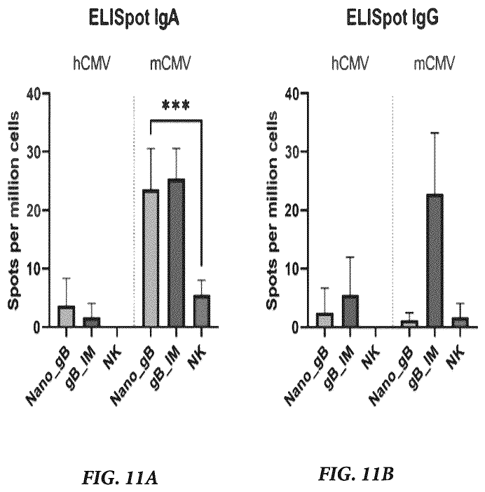

The result shows a stronger response when mCMV gB is administered, especially the IgA class. A statistically significant difference was achieved not only for the intramuscular administration of the mentioned variant of gB, but also in the case of the used nanomaterial containing gB. The hCMV variant then showed a weak response in both monitored classes of antibodies. FIGS. 11A & 11B is a graph illustrating detection of antibody-producing cells isolated from the spleens of immunized animals.

Spleens were collected after animal sacrifice on 42^{nd} day from first immunization. Splenocytes were subsequently isolated from the removed spleens, which, after stimulation in a medium containing gB, were subjected to analysis on a flow cytometer and a test for the detection of cells secreting antibodies (B-cell ELISpot) and cytokines (T-cell ELISpot). Cell markers CD3 (T-lymphocytes), CD4 (marker of Th), CD8 (marker of Tc) and especially cell memory (CD44 and CD62L) were monitored on a flow cytometer. The expression of CD44 and CD62L were then determined for these subsets.

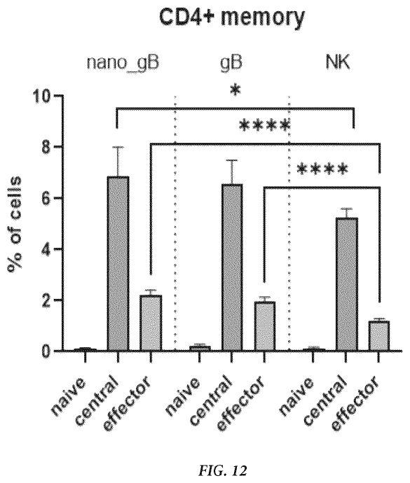

By monitoring CD3, CD4 and CD8 cell populations and especially the expression of surface markers associated with cellular memory (CD44 and CD62L), statistically significant differences were obtained between NK and animals immunized with gB_IM and especially nano_gB, when in the case of using mCMV gB, differences were found in cells expressing markers central and effector memories for the CD4+ population. FIG. 12 shows cellular memory for Th lymphocytes (CD4 is the marker for Th lymphocytes) when mCMV gB is used for immunization of mice, with animals receiving intramuscular injection of mCMV gB (shown as gB on FIG. 12) and animals receiving oral thin films hCMV gB (shown as nano_gB on FIG. 12) have CD4+ memory at a statistically higher level than animals not receiving any immunization.

The pharmaceutical composition for oromucosal administration, the oral thin film produced by electrospinning, particularly for vaccination against cytomegalovirus infections, can be used as a preventive agent or as a therapeutic agent against cytomegalovirus infections, particularly in immunosuppressed patients.

In a first embodiment, a pharmaceutical composition for oromucosal administration is provided, the pharmaceutical composition comprises: at least one coating layer comprising a water-insoluble, pharmaceutically acceptable polymer, and a conductive agent; an active layer comprising a water-soluble and pharmaceutically acceptable polymer, a conductive agent, and cytomegalovirus glycoprotein B as the active ingredient, wherein the active layer is flexible and moldable.

In a second embodiment, the first embodiment includes cytomegalovirus glycoprotein B is present at 2 to 250 μg.

In a third embodiment, each of the first and second embodiment includes the active layer comprises polymeric nanofiber formed by electrospinning.

In a fourth embodiment, the third embodiment includes cytomegalovirus glycoprotein B is releasably deposited in the polymer nanofiber.

In a fifth embodiment, each of the first and second embodiment includes the at least one coating layer comprises polymeric nanofibers formed by electrospinning.

In a sixth embodiment, each of the first, second, and fourth embodiment includes the conductive agent is an acid, a base, a neutral salt, an acidic salt, or a basic salt.

In a seventh embodiment, the sixth embodiment includes the acid is phosphoric acid, formic acid, acetic acid, citric acid, lactic acid, acetic acid, ascorbic acid, malic acid, gluconic acid, glutamic acid, hydrochloric acid, sulfuric acid, succinic acid, tartaric acid, or butyric acid.

In an eighth embodiment, the sixth embodiment includes the base is sodium hydroxide or potassium hydroxide.

In a ninth embodiment, the sixth embodiment includes the neutral salt is sodium chloride, potassium chloride, arginine hydrochloride, triethanolamine, or tetraethylammonium bromide.

In a tenth embodiment, the sixth embodiment includes the acidic salt is sodium hydrogen sulfate, mono- or di-potassium sulfate, or mono- or di-sodium phosphate.

In an eleventh embodiment, the sixth embodiment includes the basic salt is sodium (bi) carbonate or sodium acetate.

In a twelfth embodiment, each of the first, second, fourth, seventh, and eleventh embodiment includes water soluble and pharmaceutically acceptable polymer is polyethylene glycols (PEG), polyethylene oxides (PEO), copolymers of polyethylene oxide with other alkylene oxides, copolymers of PEG and polypropylene oxide or polybutylene oxide, polyvinyl alcohol, polyvinylpyrrolidones (PVP), albumin, dextran, hyaluronic acid, alginates, carrageenan, chitosan, gelatin, collagen, zein and derivatives, polyacrylic acid, cellulose derivatives such as sodium carboxymethyl cellulose, hydroxyethyl cellulose (HEC), hydroxypropyl cellulose (HPC), hydroxypropyl methylcellulose (HPMC), hydroxypropyl methylcellulose phthalate, cellulose acetate phthalate, or oxidized cellulose.

In a thirteenth embodiment, each of the first, second, fourth, seventh, and eleventh embodiment includes water insoluble and pharmaceutically acceptable polymer is acetylcellulose, methylcellulose, ethylcellulose, noncrystalline cellulose, acrylates, methacrylates, alkylacrylates and copolymers thereof, (meth)acrylic acid, (meth)acrylamides, hydroxyethyl (meth)acrylates, poly(alpha-hydroxy acids), poly(c-caprolactone), poly(lactide-co-glycolide), poly(alpha-aminoacids) and its copolymers, or polyurethane.

In a fourteenth embodiment, each of the first, second, fourth, seventh, and eleventh embodiment includes water-insoluble and pharmaceutically acceptable polymer is a biodegradable polymer.

In a fifteenth embodiment, the fourteenth embodiment includes the biodegradable polymer is polycaprolactone, polylactic acid, poly(lactide-co-glycolide), or polyglycolic acid.

In a sixteenth embodiment, each of the first to fifteenth embodiments includes the pharmaceutical composition further comprises a wetting agent or a penetration enhancer.

In a seventeenth embodiment, the sixteenth embodiment includes the wetting agent or penetration enhancer is anionic surfactants, cationic surfactants, ionic surfactants, fatty acids and derivatives, sulfoxides, alcohols, chelating agents, bile salts and derivatives, cyclodextrins, polyvinylpyrrolidone, lactose, triacetin, and/or menthol.

In an eighteenth embodiment, a use of the pharmaceutical composition of the first embodiment in the prevention of cytomegalovirus infections is provided.

In a nineteenth embodiment, a use of the pharmaceutical composition of the first embodiment in the treatment of cytomegalovirus infections is provided.

In a twentieth embodiment, each of the eighteenth and nineteenth embodiment includes the pharmaceutical composition is administered to an oromucosal membrane of a subject.

In a twenty first embodiment, the eighteenth embodiment includes administering to a subject with a prime administration followed by two booster administrations.

In a twenty second embodiment, the twenty first embodiment includes the prime administration is administered on day 1, the two booster administrations are administered on day 14 and day 28.

In a twenty third embodiment, the twenty first embodiment includes the prime administration is administered on day 1, followed by first booster on at least 7 days thereafter, followed by second booster on at least 7 days thereafter.

In a twenty fourth embodiment, the eighteenth embodiment includes the pharmaceutical composition of claim the first embodiment is administered to a subject with a prime administration followed by at least one booster administration, and each booster administration is on at least 7 days after the prior administration.

In a twenty fifth embodiment, a use of cytomegalovirus glycoprotein B for the production of a vaccine for oromucosal vaccination against cytomegalovirus infections is provided.

In a twenty sixth embodiment, a method to manufacture a pharmaceutical composition for oromucosal administration is provided, the method comprises: combining a water insoluble and pharmaceutically acceptable polymer and a conductive agent into water to form a first solution; electrospinning the first solution; collecting nanofibers produced from electrospinning of the first solution on a mat, the collected nanofibers forming a coating layer; combining cytomegalovirus glycoprotein B, a water soluble and pharmaceutically acceptable polymer, and a conductive agent into water to form a second solution; electrospinning the second solution; and collecting nanofibers produced from electrospinning of the second solution on top of the coating layer.

In a twenty seventh embodiment, the twenty sixth embodiment includes the water insoluble and pharmaceutically acceptable polymer is acetylcellulose, methyl cellulose, ethylcellulose, noncrystalline cellulose, acrylates, methacrylates, alkylacrylates and copolymers thereof, (meth)acrylic acid, (meth)acrylamides, hydroxyethyl (meth)acrylates, poly(alpha-hydroxy acids), poly(c-caprolactone), poly(lactide-co-glycolide), poly(alpha-aminoacids) and its copolymers, or polyurethanes.

In a twenty eighth embodiment, the twenty sixth embodiment includes the water soluble and pharmaceutically acceptable polymer is polyethylene glycols (PEG), polyethylene oxides (PEO), copolymers of polyethylene oxide with other alkylene oxides, copolymers of PEG and polypropylene oxide or polybutylene oxide, polyvinyl alcohol, polyvinylpyrrolidones (PVP), albumin, dextran, hyaluronic acid, alginates, carrageenan, chitosan, gelatin, collagen, zein and derivatives, polyacrylic acid, cellulose derivatives such as sodium carboxymethyl cellulose, hydroxyethyl cellulose (HEC), hydroxypropyl cellulose (HPC), hydroxypropyl methyl cellulose (HPMC), hydroxypropyl methyl cellulose phthalate, cellulose acetate phthalate, or oxidized cellulose.

In a twenty ninth embodiment, each of the twenty sixth to twenty seventh embodiments includes the conductive agent is an acid, a base, a neutral salt, an acidic salt, or a basic salt.

In a thirtieth embodiment, the twenty ninth embodiment includes the acid is phosphoric acid, formic acid, acetic acid, citric acid, lactic acid, acetic acid, ascorbic acid, malic acid, gluconic acid, glutamic acid, hydrochloric acid, sulfuric acid, succinic acid, tartaric acid, or butyric acid.

In a thirty first embodiment, the twenty ninth embodiment includes the base is sodium hydroxide or potassium hydroxide.

In a thirty second embodiment, the twenty ninth embodiment includes the neutral salt is sodium chloride, potassium chloride, arginine hydrochloride, triethanolamine, or tetraethylammonium bromide.

In a thirty third embodiment, the twenty ninth embodiment includes the acidic salt is sodium hydrogen sulfate, mono- or di-potassium sulfate, mono- or di-sodium phosphate.

In a thirty fourth embodiment, the twenty ninth embodiment includes the basic salt is sodium (bi) carbonate or sodium acetate.

In a thirty fifth embodiment, the twenty sixth embodiment further comprises combining a wetting agent and/or a penetration enhancer into the first and/or the second solution.

In a thirty sixth embodiment, the thirty fifth embodiment includes the wetting agent or penetration enhancer is anionic surfactants, cationic surfactants, ionic surfactants, fatty acids and derivatives, sulfoxides, alcohols, chelating agents, bile salts and derivatives, cyclodextrins, polyvinylpyrrolidone, lactose, triacetin, and/or menthol.

In a thirty seventh embodiment, a solution for electrospinning is provided, the solution comprises: a water soluble and pharmaceutically acceptable polymer, a conductive agent, and cytomegalovirus glycoprotein B.

In a thirty eighth embodiment, the thirty seventh embodiment includes the water insoluble and pharmaceutically acceptable polymer is acetylcellulose, methylcellulose, ethylcellulose, noncrystalline cellulose, acrylates, methacrylates, alkylacrylates and copolymers thereof, (meth)acrylic acid, (meth)acrylamides, hydroxyethyl (meth)acrylates, poly(alpha-hydroxy acids), poly(c-caprolactone), poly(lactide-co-glycolide), poly(alpha-aminoacids) and its copolymers, or polyurethane.

In a thirty ninth embodiment, the thirty seventh embodiment includes the water soluble and pharmaceutically acceptable polymer is polyethylene glycols (PEG), polyethylene oxides (PEO), copolymers of polyethylene oxide with other alkylene oxides, copolymers of PEG and polypropylene oxide or polybutylene oxide, polyvinyl alcohol, polyvinylpyrrolidones (PVP), albumin, dextran, hyaluronic acid, alginates, carrageenan, chitosan, gelatin, collagen, zein and derivatives, polyacrylic acid, cellulose derivatives such as sodium carboxymethyl cellulose, hydroxyethyl cellulose (HEC), hydroxypropyl cellulose (HPC), hydroxypropyl methylcellulose (HPMC), hydroxypropyl methylcellulose phthalate, cellulose acetate phthalate, or oxidized cellulose.

In a fortieth embodiment, each of the thirty seventh to thirty ninth embodiment includes the conductive agent is an acid, a base, a neutral salt, an acidic salt, or a basic salt.

In a forty first embodiment, the fortieth embodiment includes the acid is phosphoric acid, formic acid, acetic acid, citric acid, lactic acid, acetic acid, ascorbic acid, malic acid, gluconic acid, glutamic acid, hydrochloric acid, sulfuric acid, succinic acid, tartaric acid, or butyric acid.

In a forty second embodiment, the fortieth embodiment includes the base is sodium hydroxide or potassium hydroxide.

In a forty third embodiment, the fortieth embodiment includes the neutral salt is sodium chloride, potassium chloride, arginine hydrochloride, triethanolamine, or tetraethylammonium bromide.

In a forty fourth embodiment, the fortieth embodiment includes the acidic salt is sodium hydrogen sulfate, mono- or di-potassium sulfate, mono- or di-sodium phosphate.

In a forty fifth embodiment, the fortieth embodiment includes the basic salt is sodium (bi) carbonate or sodium acetate.

In a forty sixth embodiment, the fortieth embodiment further comprises a wetting agent and/or a penetration enhancer.

In a forty seventh embodiment, the forty sixth embodiment includes the wetting agent or penetration enhancer is anionic surfactants, cationic surfactants, ionic surfactants, fatty acids and derivatives, sulfoxides, alcohols, chelating agents, bile salts and derivatives, cyclodextrins, polyvinylpyrrolidone, lactose, triacetin, and/or menthol.
[1] V. Chvátalová, B. Šebánková, H. Hrbáčková, P. Tureček, and J. Flegr, “Differences in cognitive functions between cytomegalovirus-infected and cytomegalovirus-free university students. Sci. Rep., vol. 8, no. 1, p. 5322, March 2018, doi: 10.1038/s41598-018-23637-3.
[2] A. S. T. Kutz, E. Muhl, H. Hackstein, H. Kirchner, and G. Bein, “High Incidence of Active Cytomegalovirus Infection Among Septic Patients,” Clin. Infect. Dis., vol. 26, no. 5, pp. 1076-1082, May 1998, doi: 10.1086/520307.
[3] E. Gane et al., “Randomised trial of efficacy and safety of oral ganciclovir in the prevention of cytomegalovirus disease in liver-transplant recipients. The Oral Ganciclovir International Transplantation Study Group [corrected]., “Lancet (London, England), vol. 350, no. 9093, pp. 1729-1733, December 1997, doi: 10.1016/s0140-6736(97)05535-9.
[4] D. Lowance et al. “Valacyclovir for the prevention of cytomegalovirus disease after renal transplantation. International Valacyclovir Cytomegalovirus Prophylaxis Transplantation Study Group,” N. Engl. J. Med., vol. 340, no. 19, pp. 1462-1470, May 1999, doi: 10.1056/NEJM199905133401903.
[5] S. Chandramouli et al., “Structure of HCMV glycoprotein B in the postfusion conformation bound to a neutralizing human antibody,” Nat. Commun. vol. 6, no. 1, p. 8176, 2015, doi: 10.1038/ncomms9176.
[6] J. JIRSAK, Oldrich; SANETRNIK, Filip; LUKAS, David; KOTEK, Vaclav; MARTINOVA, Lenka; CHALOUPEK, “METHOD OF NANOFIBRES PRODUCTION FROM A POLYMER SOLUTION USING ELECTROSTATIC SPINNING AND A DEVICE FOR CARRYING OUT THE METHOD,” 2005 doi: WO2005/024101.

Citation of the above publications or documents is not intended as an admission that any of the foregoing is pertinent prior art, nor does it constitute any admission as to the contents or date of these publications or documents.

1. A pharmaceutical composition for oromucosal administration, comprising:
at least one coating layer comprising a water insoluble, pharmaceutically acceptable polymer, and a conductive agent, wherein the at least one coating layer is formulated as polymeric nanofibers formed by electrospinning,
an active layer comprising a water soluble and pharmaceutically acceptable polymer, a conductive agent, and cytomegalovirus glycoprotein B as the active ingredient, wherein the at least one active layer is formulated as polymeric nanofibers formed by electrospinning wherein the active layer is flexible and moldable.

2. The pharmaceutical composition of claim 1, wherein cytomegalovirus glycoprotein B is present at 2 to 250 μg.

3-5. (canceled)

6. The pharmaceutical composition of claim 1, wherein the conductive agent is an acid, a base, a neutral salt, an acidic salt, or a basic salt.

7. The pharmaceutical composition of claim 6, wherein the acid is phosphoric acid, formic acid, acetic acid, citric acid, lactic acid, acetic acid, ascorbic acid, malic acid, gluconic acid, glutamic acid, hydrochloric acid, sulfuric acid, succinic acid, tartaric acid, or butyric acid.

8. The pharmaceutical composition of claim 6, wherein the base is sodium hydroxide or potassium hydroxide.

9. The pharmaceutical composition of claim 6, wherein the neutral salt is sodium chloride, potassium chloride, arginine hydrochloride, triethanolamine, or tetraethylammonium bromide.

10. The pharmaceutical composition of claim 6, wherein the acidic salt is sodium hydrogen sulfate, mono- or di-potassium sulfate, or mono- or di-sodium phosphate.

11. The pharmaceutical composition of claim 6, wherein the basic salt is sodium (bi) carbonate or sodium acetate.

12. The pharmaceutical composition of claim 1, wherein water soluble and pharmaceutically acceptable polymer comprises three or more of polyethylene glycols (PEG), polyethylene oxides (PEO), copolymers of polyethylene oxide with other alkylene oxides, copolymers of PEG and polypropylene oxide or polybutylene oxide, polyvinyl alcohol, polyvinylpyrrolidones (PVP), albumin, dextran, hyaluronic acid, alginates, carrageenan, chitosan, gelatin, collagen, zein and derivatives, polyacrylic acid, cellulose derivatives such as sodium carboxymethyl cellulose, hydroxyethyl cellulose (HEC), hydroxypropyl cellulose (HPC), hydroxypropyl methylcellulose (HPMC), hydroxypropyl methylcellulose phthalate, cellulose acetate phthalate, or oxidized cellulose.

13. The pharmaceutical composition of claim 1, wherein water insoluble and pharmaceutically acceptable polymer is acetylcellulose, methylcellulose, ethylcellulose, noncrystalline cellulose, acrylates, methacrylates, alkylacrylates and copolymers thereof, (meth)acrylic acid, (meth)acrylamides, hydroxyethyl (meth)acrylates, poly(alpha-hydroxy acids), poly(c-caprolactone), poly(lactide-co-glycolide), poly(alpha-aminoacids) and its copolymers, or polyurethane.

14. The pharmaceutical composition of claim 1, wherein water-insoluble and pharmaceutically acceptable polymer is a biodegradable polymer, and the biodegradable polymer is polycaprolactone, polylactic acid, poly(lactide-co-glycolide), or polyglycolic acid.

15. (canceled)

16. The pharmaceutical composition of claim 1, further comprising a wetting agent or a penetration enhancer, and the wetting agent or penetration enhancer is an anionic surfactant, a cationic surfactant, an ionic surfactant, a fatty acid or derivative thereof, a sulfoxide, an alcohol, a chelating agent, a bile salt or derivative thereof, a cyclodextrin, polyvinylpyrrolidine, lactose, triacetin, and/or menthol.

17. (canceled)

18. Use of the pharmaceutical composition of claim 1 in the prevention or treatment of cytomegalovirus infections.

19. (canceled)

20. The use of claim 18, wherein the pharmaceutical composition is administered to an oromucosal membrane of a subject.

21. The use of claim 18, wherein the pharmaceutical composition is administered to a subject with a prime administration followed by two booster administrations.

22. (canceled)

23. (canceled)

24. The use of claim 18, wherein the pharmaceutical is administered to a subject with a prime administration followed by at least one booster administration, and each booster administration is on at least 7 days after the prior administration.

25. (canceled)

26. A method of manufacturing the pharmaceutical composition of claim 1, comprising:
combining a water-insoluble and pharmaceutically acceptable polymer and a conductive agent into water to form a first solution;
electrospinning the first solution by needless electrospinning;
collecting nanofibers produced from electrospinning of the first solution on a mat, the collected nanofibers forming a coating layer;
combining cytomegalovirus glycoprotein B, a water soluble and pharmaceutically acceptable polymer, and a conductive agent into water to form a second solution;
electrospinning the second solution; and
collecting nanofibers produced from electrospinning of the second solution on top of the coating layer.

27. The method of claim 26, wherein:
the water insoluble and pharmaceutically acceptable polymer is acetylcellulose, methylcellulose, ethylcellulose, noncrystalline cellulose, acrylates, methacrylates, alkylacrylates and copolymers thereof, (meth)acrylic acid, (meth)acrylamides, hydroxyethyl (meth)acrylates, poly(alpha-hydroxy acids), poly(c-caprolactone), poly(lactide-co-glycolide), poly(alpha-aminoacids) and its copolymers, or polyurethanes;
the water soluble and pharmaceutically acceptable polymer comprises three or more of is polyethylene glycols (PEG), polyethylene oxides (PEO), copolymers of polyethylene oxide with other alkylene oxides, copolymers of PEG and polypropylene oxide or polybutylene oxide, polyvinyl alcohol, polyvinylpyrrolidones (PVP), albumin, dextran, hyaluronic acid, alginates, carrageenan, chitosan, gelatin, collagen, zein and derivatives, polyacrylic acid, cellulose derivatives such as sodium carboxymethyl cellulose, hydroxyethyl cellulose (HEC), hydroxypropyl cellulose (HPC), hydroxypropyl methylcellulose (HPMC), hydroxypropyl methylcellulose phthalate, cellulose acetate phthalate, or oxidized cellulose;
the conductive agent is an acid, a base, a neutral salt, an acidic salt, or a basic salt; or
the method further comprise, combining a wetting agent and/or a penetration enhancer into the first and/or the second solution, and the wetting agent or penetration enhancer is anionic surfactants, cationic surfactants, ionic surfactants, fatty acids and derivatives, sulfoxides, alcohols, chelating agents, bile salts and derivatives, cyclodextrins, polyvinylpyrrolidone, lactose, triacetin, and/or menthol;

28-30. (canceled)

31. The method of claim 27, wherein the base is sodium hydroxide or potassium hydroxide.

32-36. (canceled)

37. A solution for electrospinning, comprising a solution comprising the pharmaceutical composition of claim 1.

38-47. (canceled)
